(19) United States
(12) Patent Application Publication
(10) Pub. No.: US 2024/0059961 A1
(43) Pub. Date: Feb. 22, 2024

(54) BIOCOMPATIBLE OXYGEN-SENSITIVE MATERIALS

(71) Applicant: Promega Corporation, Madison, WI (US)

(72) Inventors: Ian Marozas, San Luis Obispo, CA (US); Zhiyang Zeng, Marshall, WI (US); Wenhui Zhou, Madison, WI (US); James J. Cali, Madison, WI (US); Mike Valley, Madison, WI (US); Jolanta Vldugiriene, Madison, WI (US)

(21) Appl. No.: 18/447,907

(22) Filed: Aug. 10, 2023

Related U.S. Application Data
(60) Provisional application No. 63/397,209, filed on Aug. 11, 2022.

(51) Int. Cl. C09K 11/06; C07D 417/04; C07F 15/00; C08G 77/04; G01N 21/64; G01N 33/497

(52) U.S. Cl. CPC C09K 11/06; C07D 417/04; C07F 15/0086; C08G 77/04; G01N 21/6428; G01N 33/497; C09K 2211/185; G01N 2021/6439

(57) ABSTRACT

Provided herein are polysiloxane matrices and biocompatible films thereof for the detection of oxygen. In particular, oxygen-sensitive fluorophores embedded within polysiloxane matrices are utilized for detection of oxygen consumption rate in living cells.

### CROSS-REFERENCE TO RELATED APPLICATIONS

This application claims the benefit of U.S. Provisional Patent Application No. 63/397,209, filed on Aug. 11, 2022, which is incorporated by reference herein.

### FIELD

Provided herein are polysiloxane matrices and biocompatible films thereof for the detection of oxygen. In particular, oxygen-sensitive fluorophores embedded within polysiloxane matrices are utilized for detection of oxygen consumption rate in living cells.

### BACKGROUND

Cells consume oxygen as they respirate, and the consumption rate of oxygen can be used to characterize the cellular metabolic phenotype. Currently, the most popular method of oxygen consumption measurements is with a standalone device and specialty equipment.

### SUMMARY

Provided herein are polysiloxane matrices and biocompatible films thereof for the detection of oxygen. In particular, oxygen-sensitive fluorophores embedded within polysiloxane matrices are utilized for detection of oxygen consumption rate in living cells.

In some embodiments, provided herein are polysiloxane matrices comprising:

(a) a bis(trialkoxysilyl) monomer of formula (I):

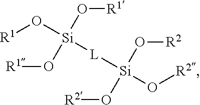

wherein R¹, R^{1′}, R^{1″}, R², R^{2′}, and R^{2″} are independently selected from CH₃, CH₂CH₃, (CH₂)₂CH₃, wherein L is selected from C₁-C₆ alkyl, C₁-C₆ heteroalkyl, C₂-C₆ alkenyl, C₂-C₆ heteroalkenyl, C₂-C₆ alkynyl, C₁-C₆ heteroalkynyl,

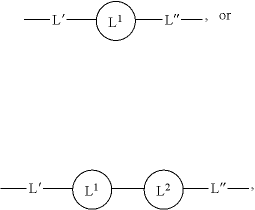

wherein L′ and L″ are independently selected from, a covalent bond, C₁-C₆ alkyl or heteroalkyl, C₂-C₆ alkenyl or heteroalkenyl, C₂-C₆ alkynyl or heteroalkynyl, wherein L¹ and L² are independently selected aryl or heteroaryl rings, wherein L¹ and L² are linked by a covalent bond, and wherein L, L′, L″, and L¹ and L² are optionally branched or substituted;

(b) a dialkyldialkoxysilane monomer of formula (II):

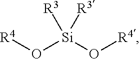

wherein R³, R^{3′}, R⁴, and R^{4′} are independently selected from CH₃, CH₂CH₃, and (CH₂)₂CH₃. In some embodiments, (i) each of R¹, R^{1′}, R^{1″}, R², R^{2′}, and R^{2″} are the same; (ii) L′ and L″, when present, are the same; (iii) L¹ and L², when present, are the same; (iv) R³ and R^{3′} are the same; and/or (v) R⁴ and R^{4″} are the same. In some embodiments, R¹, R^{1′}, R^{1″}, R², R^{2′}, and R^{2″} are CH₃ or CH₂CH₃. In some embodiments, L is selected from (CH₂)₁₋₆, (CH₂)₁₋₆—NH—(CH₂)₁₋₆, (CH₂)₁₋₆—O—(CH₂)₁₋₆, (CH₂)₀₋₆-aryl-(CH₂)₁₋₆, and (CH₂)₁₋₆-aryl-aryl-(CH₂)₁₋₆. In some embodiments, the bis(trialkoxysilyl) monomer is selected from 1,4-bis(trimethoxysilylethyl)benzene, 1,4-bis(trimethoxysilylmethyl)benzene, 1,4-bis(trimethoxysilyl)benzene, 4,4′-bis(triethoxysilyl)-1,1′-biphenyl, 1,2-bis(triethoxysilyl)ethane, and bis[3-(trimethoxysilyl)propyl]amine. In some embodiments, R³, R^{3′}, R⁴, R^{4′} are CH₃ or CH₂CH₃. In some embodiments, the bis(trialkoxysilyl) monomer is 1,4-bis(trimethoxysilylethyl)benzene and the dialkyldialkoxysilane monomer is dimethyldimethoxysilane.

In some embodiments, provided herein is a composition comprising a polysiloxane matrix as described herein and an oxygen-sensitive fluorophore. In some embodiments, the oxygen-sensitive fluorophore is selected from tris(4,7-diphenyl-1,10-phenanthroline) ruthenium(II) chloride (Ru-dpp), Platinum octaethylporphyrin; platinum(II) 2,3,7,8,12,13,17,18-octaethyl-21H,23H-porphyrin (PtOEP), Palladium(II) octaethylporphine (PdOEP), Platinum(II)-5,10,15,20-tetrakis-(2,3,4,5,6-pentafluorphenyl)-porphyrin (PtTfPP), palladium(II)-5,10,15,20-tetrakis-(2,3,4,5,6-pentafluorphenyl)-porphyrin (PdTFPP), platinum(II) octaethylporphyrinketone (PtOEPK), palladium(II) octaethylporphyrinketone (PdOEPK), platinum(II) tetraphenyltetrabenzoporphyrin (PtTPTBP), meso-Tetraphenyl-tetrabenzoporphine Palladium Complex (PdTPTBP), platinum(II) tetraphenyltetranaphthoporphyrin (PtTPTNP), and palladium(II) tetraphenyltetranaphthoporphyrin (PdTPTNP).

In some embodiments, compositions further comprise an oxygen-insensitive fluorophore. In some embodiments, the oxygen-insensitive fluorophore is selected from Nile blue chloride, tris(8-hydroxyquinolinato)aluminum (AlQ3), TAMRA, and Coumarin-6. In some embodiments, the oxygen-insensitive fluorophore is Coumarin-6 and the oxygen-sensitive fluorophore is PtTfPP.

In some embodiments, provided herein are thin films comprising the polysiloxane matrices (e.g., with embedded fluorophore(s)) described herein.

In some embodiments, provided herein are devices comprising a solid surface with a polysiloxane matrix thin film described herein (with embedded fluorophore(s)) deposited thereupon. In some embodiments, the solid surface is a glass or plastic (e.g., polystyrene) surface. In some embodiments, the solid surface is a slide or the bottom of a microwell. In some embodiments, devices further comprise a layer of bioactive extracellular matrix protein or biocompatible molecules on top of the thin film. In some embodiments, the layer of bioactive extracellular matrix protein comprises one or more proteins selected from type I collagen, type IV collagen, fibronectin, and laminin. In some embodiments, the layer of biocompatible molecules comprises polydopamine. In some embodiments, the bioactive extracellular matrix protein is passively coated onto the thin film. In some embodiments, the bioactive extracellular matrix protein is covalently or non-covalently attached to the thin film.

In some embodiments, provided herein are methods comprising: (a) contacting (i) an oxygen-sensitive fluorophore embedded within a polysiloxane-matrix with (ii) a test solution; (b) exposing the oxygen-sensitive fluorophore to light within the excitation spectrum of the oxygen-sensitive fluorophore; (c) detecting light within the emission spectrum of the oxygen-sensitive fluorophore; (d) determining the concentration of oxygen in the test solution. In some embodiments, the polysiloxane-matrix determining the concentration of oxygen in the test solution comprises comparing the light within the emission spectrum of the oxygen-sensitive fluorophore to reference values. In some embodiments, the reference values are the amount of light emitted when the test solution is saturated with oxygen and the amount of light emitted when the test solution is depleted of oxygen. In some embodiments, the reference values are the amount of light emitted at varying oxygen concentrations. In some embodiments, the test solution comprises cells. In some embodiments, steps (b)-(d) are repeated to monitor the consumption of oxygen by the cells over time. In some embodiments, the polysiloxane-matrix is a film deposited on a transparent solid surface. In some embodiments, the solid surface is glass or plastic (e.g., polystyrene). In some embodiments, the solid surface is a slide or the bottom of a microwell. In some embodiments, the film further comprises a layer of bioactive extracellular matrix protein or biocompatible molecules deposited thereon. In some embodiments, the layer of bioactive extracellular matrix protein comprises one or more proteins selected from type I collagen, type IV collagen, fibronectin, and laminin. In some embodiments, the layer of biocompatible molecules comprises polydopamine.

In some methods, also embedded within the polysiloxane-matrix is an oxygen-insensitive fluorophore. In some embodiments, oxygen-insensitive fluorophore is selected from Nile blue chloride, tris(8-hydroxyquinolinato)aluminum (AlQ3), TAMRA, and Coumarin-6. In some embodiments, methods further comprise steps of exposing the oxygen-insensitive fluorophore to light within the excitation spectrum of the oxygen-insensitive fluorophore and detecting light within the emission spectrum of the oxygen-insensitive fluorophore. In some embodiments, determining the concentration of oxygen in the test solution comprises comparing reference values to the ratio of (i) the light within the emission spectrum of the oxygen-sensitive fluorophore over (ii) the light within the emission spectrum of the oxygen-insensitive fluorophore. In some embodiments, the reference values are the ratio of light emitted when the test solution is saturated with oxygen and the ratio of light emitted when the test solution is depleted of oxygen. In some embodiments, the reference values are the ratio of light emitted at varying oxygen concentrations.

In some methods, the polysiloxane matrix comprises: (a) a bis(trialkoxysilyl) monomer of formula (I):

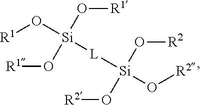

wherein R¹, R^{1′}, R_{1″}, R², R^{2′}, and R^{2″} are independently selected from CH₃, CH₂CH₃, (CH₂)₂CH₃, wherein L is selected from C₁-C₆ alkyl, C₁-C₆ heteroalkyl, C₂-C₆ alkenyl, C₂-C₆ heteroalkenyl, C₂-C₆ alkynyl, C₁-C₆ heteroalkynyl,

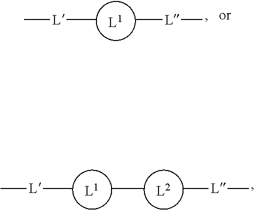

wherein L′ and L″ are independently selected from, a covalent bond, C₁-C₆ alkyl or heteroalkyl, C₂-C₆ alkenyl or heteroalkenyl, C₂-C₆ alkynyl or heteroalkynyl, wherein L¹ and L² are independently selected aryl or heteroaryl rings, wherein L¹ and L² are linked by a covalent bond, and wherein L, L′, L″, and L¹ and L² are optionally branched or substituted; (b) a dialkyldialkoxysilane monomer of formula (II):

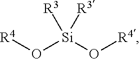

wherein R³, R^{3′}, R⁴, and R^{4′} are independently selected from CH₃, CH₂CH₃, and (CH₂)₂CH₃. In some embodiments, (i) each of R¹, R^{1′}, R^{1″}, R², R^{2′}, and R^{2″} are the same; (ii) L′ and L″, when present, are the same; (iii) L¹ and L², when present, are the same; (iv) R³ and R^{3′} are the same; and/or (v) R⁴ and R^{4″} are the same. In some embodiments, R¹, R^{1′}, R^{1″}, R², R^{2′}, and R^{2″} are CH₃ or CH₂CH₃. In some embodiments, L is selected from (CH₂)₁₋₆, (CH₂)₁₋₆—NH—(CH₂)₁₋₆, (CH₂)₁₋₆—O—(CH₂)₁₋₆, (CH₂)₀₋₆-aryl-(CH₂)₁₋₆, and (CH₂)₁₋₆-aryl-aryl-(CH₂)₁₋₆. In some embodiments, the bis(trialkoxysilyl) monomer is selected from 1,4-bis(trimethoxysilylethyl)benzene, 1,4-bis(trimethoxysilylmethyl)benzene, 1,4-bis(trimethoxysilyl)benzene, 4,4′-bis(triethoxysilyl)-1,1′-biphenyl, 1,2-bis(triethoxysilyl)ethane, and bis[3-(trimethoxysilyl)propyl]amine. In some embodiments, R³, R^{3′}, R⁴, R^{4′} are CH₃ or CH₂CH₃. In some embodiments, the bis(trialkoxysilyl) monomer is 1,4-bis(trimethoxysilylethyl)benzene and the dialkyldialkoxysilane monomer is dimethyldimethoxysilane. In some embodiments, the oxygen-sensitive fluorophore is selected from Ru-dpp, PtOEP, PdOEP, PtTfPP, PdTFPP, PtOEPK, PdOEPK, PtTPTBP, PdTPTBP, PtTPTNP, and PdTPTNP.

### BRIEF DESCRIPTION OF THE DRAWINGS

FIG. 1. Schematic of an exemplary embodiment. An oxygen sensitive dye, PtTFPP, is immobilized in a cellular substrate. The dye is highly fluorescent at low oxygen concentrations; as cells consume oxygen, the concentration of oxygen drops, thereby increasing the fluorescence of the sensor dye. The fluorescence of the dye is monitored by excitation with a plate reader from the bottom of the substrate.

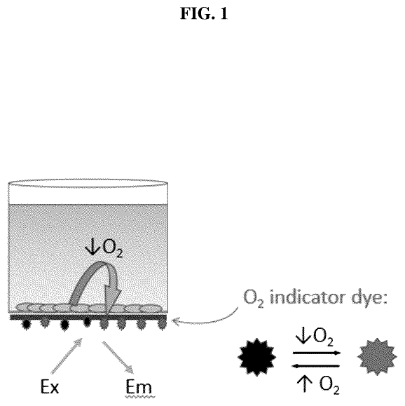

FIG. 2. Components of an exemplary oxygen sensor matrix.

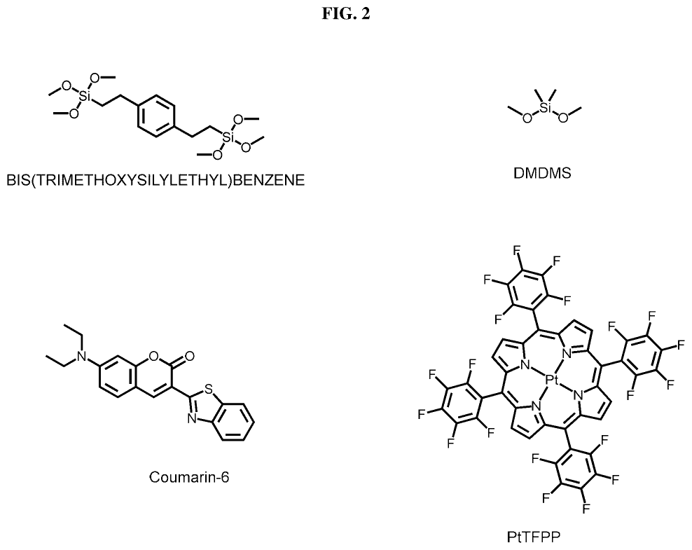

FIG. 3. Image of an exemplary sensor matrix coating the bottom of a 96 well plate.

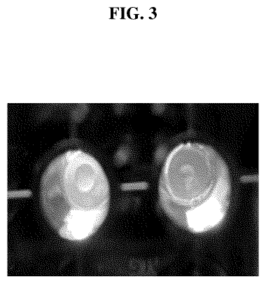

FIG. 4. Oxygen concentration changes with 0.05 u-1 u/well glucose oxidase in wells of a 96-well plate coated with an exemplary oxygen sensing film.

FIG. 5. HCT-116 titration from 0-120,000 cells/well. Oxygen concentration decreased over time relative to a cellular control in a cell-density dependent manner (n=16 replicates).

FIG. 6. HCT-116 treatment with FCCP and Rotenone. Average oxygen concentration decreased over time relative to a cellular control in dose dependent manner with FCCP (n=16 replicates). Change in oxygen concentration over 30 minutes shows trend in oxygen consumption rate with FCCP treatment (n=16). Change in oxygen concentration over 30 minutes with Rotenone (n=8).

FIG. 7. HEP-G2 titration from 0-120,000 cells/well (n=4 replicates) with and without rotenone (2 uM).

FIG. 8. PC-3 titration from 0-100,000 cells/well (n=4 replicates). Change of oxygen concentration for PC-3 after FCCP and rotenone treatment.

FIG. 9. Comparison of system sealed with oxygen barrier (Silver Seal; Aluminum Foil) and a system open to the atmosphere. An oxygen barrier significantly increased the apparent oxygen consumption rate.

### DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” means a straight or branched saturated hydrocarbon chain containing from 1 to 30 carbon atoms, for example, 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 6 to 20 carbon atoms (C₆-C₂₀ alkyl), or 8 to 14 carbon atoms (C₈-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkylene” refers to a divalent group derived from a straight or branched chain hydrocarbon of 1 to 10 carbon atoms (C₁-C₁₀ alkylene), for example, of 1 to 6 carbon atoms (C₁-C₆ alkylene). Representative examples of alkylene include, but are not limited to, —CH₂—, —CH₂CH₂—, —CH(CH₃)—, —CH₂CH₂CH₂—, —CH₂CH(CH₃)—, —CH₂CH₂CH₂CH₂—, —CH₂CH(CH₃)CH₂—, —CH₂CH₂CH(CH₃)—, —CH₂CH₂CH₂CH₂CH₂—, —CH₂CH(CH₃)CH₂CH₂—, —CH(CH₃)CH₂CH₂CH₂—, —CH₂CH₂CH₂CH₂CH₂CH₂—, —CH₂CH₂CH(CH₃)CH₂CH₂—, —CH₂CH(CH₃)CH₂CH₂CH₂—, and —CH(CH₃)CH₂CH₂CH₂CH₂—.

As used herein, the term “alkenyl” refers to a straight or branched hydrocarbon chain containing from 2 to 30 carbon atoms and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkynyl” refers to a straight or branched hydrocarbon chain containing from 2 to 30 carbon atoms and containing at least one carbon-carbon triple bond. Representative examples of alkynyl include, but are not limited to, ethynyl, propynyl, and butynyl.

As used herein, the term “heteroalkyl” means an alkyl group, as defined herein, in which one or more of the carbon atoms (and any associated hydrogen atoms) are each independently replaced with a heteroatom group such as —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. By way of example, 1, 2 or 3 carbon atoms may be independently replaced with the same or different heteroatomic group. Examples of heteroalkyl groups include, but are not limited to, —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. Heteroalkyl also includes groups in which a carbon atom of the alkyl is oxidized (i.e., is —C(O)—).

As used herein, the term “heteroalkylene” means an alkylene group, as defined herein, in which one or more of the carbon atoms (and any associated hydrogen atoms) are each independently replaced with a heteroatom group such as —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. By way of example, 1, 2 or 3 carbon atoms may be independently replaced with the same or different heteroatomic group. Heteroalkylene also includes groups in which a carbon atom of the alkyl is oxidized (i.e., is —C(O)—). Examples of heteroalkylene groups include, but are not limited to, —CH₂—O—CH₂—, —CH₂—S—CH₂—, —CH₂—NR—CH₂—, —CH₂—NH—C(O)—CH₂, and the like, as well as polyethylene oxide chains, polypropylene oxide chains, and polyethyleneimine chains.

As used herein, the term “aryl” refers to an aromatic carbocyclic ring system having a single ring (monocyclic) or multiple rings (bicyclic or tricyclic) including fused ring systems, and zero heteroatoms. As used herein, aryl contains 6-20 carbon atoms (C₆-C₂₀ aryl), 6 to 14 ring carbon atoms (C₆-C₁₄ aryl), 6 to 12 ring carbon atoms (C₆-C₁₂ aryl), or 6 to 10 ring carbon atoms (C₆-C₁₀ aryl). Representative examples of aryl groups include, but are not limited to, phenyl, naphthyl, anthracenyl, and phenanthrenyl.

As used herein, the term “arylene” refers to a divalent aryl group. Representative examples of arylene groups include, but are not limited to, phenylene groups (e.g., 1,2-phenylene, 1,3-phenylene, and 1,4-phenylene).

As used herein, the term “heteroaryl” refers to an aromatic group having a single ring (monocyclic) or multiple rings (bicyclic or tricyclic), having one or more ring heteroatoms independently selected from O, N, and S. The aromatic monocyclic rings are five- or six-membered rings containing at least one heteroatom independently selected from O, N, and S (e.g., 1, 2, 3, or 4 heteroatoms independently selected from O, N, and S). The five-membered aromatic monocyclic rings have two double bonds, and the six- membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein, and a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— optionally also recites —OCH₂—, and —OC(O)NH— also optionally recites —NHC(O)O—.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### DETAILED DESCRIPTION

Provided herein are polysiloxane matrices and biocompatible films thereof for the detection of oxygen. In particular, oxygen-sensitive fluorophores embedded within polysiloxane matrices are utilized for detection of oxygen consumption rate in living cells.

In some embodiments, provided herein are biocompatible sensor films that allow for measurement of, oxygen content of a solution, and in particular, cellular oxygen consumption rate. In certain embodiments, the materials and systems here allow for monitoring oxygen concentration and/or rate of change thereof in a standard format (e.g., 96-well plate, 384-well plate, etc.) using standard laboratory equipment (e.g., plate reader). Some embodiments herein provide functionalized surfaces that can (a) detect the concentration of oxygen in a solution (e.g., containing cells) over time and (b) support cellular attachment.

In some embodiments, the oxygen sensing component of the film is composed of a porous, siloxane matrix impregnated with a porphyrin dye, where the fluorescence intensity changes fluorescence as a function of the oxygen concentration. An advantage of the oxygen sensitive films provided herein is the dynamic range of the film, specifically, the ratio of fluorescence intensity at hypoxic conditions divided by the fluorescence intensity at air-saturated conditions (I0/Iair). The I0/Iair ratio of exemplary materials described herein is typically 30-40, whereas other materials utilizing the same oxygen sensitive porphyrin dye are characterized by an I0/Iair ratio of 4-5. An advantage of embodiments herein is that they allow a user to measure oxygen consumption on a standard plate reader.

In some embodiments, provided herein are polysiloxane matrices comprising bis(trialkoxysilyl) and dialkyldialkoxysilane monomers with an oxygen-sensitive fluorophore embedded within the matrix. In some embodiments, a film of the matrix with the oxygen-sensitive fluorophore embedded therein is deposited as a film onto a solid substrate (e.g., glass plate, bottom of a well, etc.). In some embodiments, biocompatible and/or bioactive protein (e.g., extracellular matrix protein, etc.) or molecules (e.g., polydopamine, etc.) are layer on top of the film to facilitate cellular attachment. In some embodiments, an oxygen-insensitive fluorophore is also embedded within the matrix (e.g., to serve as a reference for the oxygen-sensitive fluorophore). In some embodiments, monitoring the fluorescence output of the oxygen-sensitive fluorophore (e.g., relative to the oxygen-insensitive fluorophore) allows detection/quantification of the oxygen content in a solution in contact with the matrix film and/or monitoring of the cellular uptake of oxygen over time.

In some embodiments, the materials and devices herein comprise a polysiloxane matrix. In some embodiments, the polysiloxane matrices herein are sufficiently transparent to allow wavelengths of light emitted from fluorophores embedded within to pass through the matrix (e.g., >50%, >60%, >70%, >80%, >90%, >95% transmittance). In some embodiments, the polysiloxane matrix is capable of adhering to glass, plastic (e.g., polystyrene), or other materials commonly used in laboratory plates, tubes, etc. In some embodiments, the polysiloxane matrices herein are biocompatible (e.g., non-toxic to cells). In some embodiments, the polysiloxane matrices herein are stable when contacted with water and non-biodegradable.

In some embodiments, the polysiloxane matrices herein a bis(trialkoxysilyl) monomer and a dialkyldialkoxysilane monomer. In some embodiments, the polysiloxane matrix is fabricated from the bis(trialkoxysilyl) and dialkyldialkoxysilane components by mixing under acidic conditions (e.g., for 10 minutes, 20 minutes, 30 minutes, 45 minutes, 1 hour, 2 hours, 3 hours, 4 hours, 6 hours, or more) followed by drying of the materials. In some embodiments, a bis(trialkoxysilyl) monomer and a dialkyldialkoxysilane monomer are mixed at a ratio of 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In some embodiments, inclusion of one or more additional components (e.g., fluorophores (e.g., oxygen-sensitive fluorophore, oxygen-insensitive fluorophore, pH-sensitive fluorophores, temperature-sensitive fluorophore, fluorophores sensitive to the concentration of specific chemical species (e.g., chemical species involved in cellular metabolism) such as reactive oxygen species (ROS), hydrogen peroxide, superoxide, nitric oxide, etc.), etc.) within the mixture allows for embedding of the additional components within he matrix and resulting film.

In some embodiments, a bis(trialkoxysilyl) monomer of the polysiloxane matrix is of formula (I):

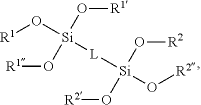

wherein R¹, R^{1′}, R^{1″}, R², R^{2′}, and R^{2″} are independently selected from CH₃, CH₂CH₃, (CH₂)₂CH₃, wherein L is selected from C₁-C₆ alkyl, C₁-C₆ heteroalkyl, C₂-C₆ alkenyl, C₂-C₆ heteroalkenyl, C₂-C₆ alkynyl, C₁-C₆ heteroalkynyl,

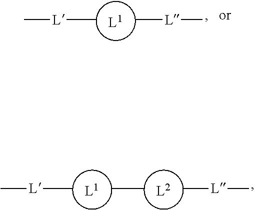

wherein L′ and L″ are independently selected from, a covalent bond, C₁-C₆ alkyl or heteroalkyl, C₂-C₆ alkenyl or heteroalkenyl, C₂-C₆ alkynyl or heteroalkynyl, wherein L¹ and L² are independently selected aryl or heteroaryl rings, wherein L¹ and L² are linked by a covalent bond. In some embodiments, each of R¹, R^{1′}, and R^{1″} are identical functional groups. In some embodiments, each of R², R^{2′}, and R^{2″} are identical functional groups. In some embodiments, each of R¹, R^{1′}, R^{1″}, R², R^{2′}, and R^{2″} are identical functional groups. In some embodiments, one or more of R¹, R^{1′}, R^{1″}, R², R^{2′}, and R^{2″} are different functional groups. In some embodiments, R¹, R^{1′}, R^{1″}, R², R^{2′}, and R^{2″} are CH₃ or CH₂CH₃. In some embodiments, L′ and L″, when present, are identical functional groups. In some embodiments, L′ and L″, when present, are different functional groups. In some embodiments, L¹ and L², when present, are identical functional groups. In some embodiments, L¹ and L², when present, are different functional groups. In some embodiments, L is selected from (CH₂)₁₋₆, (CH₂)₁₋₆—NH—(CH₂)₁₋₆, (CH₂)₁₋₆—O—(CH₂)₁₋₆, (CH₂)₀₋₆-aryl-(CH₂)₁₋₆, and (CH₂)₁₋₆-aryl-aryl- (CH₂)₁₋₆.

In some embodiments, one or more of L, L′, L″, L¹, L², R¹, R^{1′}, R^{1″}, R², R^{2′}, and R^{2″} are optionally branched or substituted. In some embodiments, one or more CH₂ groups of a L, L′, L″, L¹, L², R¹, R^{1′}, R^{1″}, R², R^{2′} and/or R^{2″} are replaced with a O, S, or NH. In some embodiments, one or more CH₃ groups of a L, L′, L″, L¹, L², R¹, R^{1′}, R^{1″}, R², R^{2′}, and/or R^{2″} are replaced with a OH, SH, or NH₂. In some embodiments, of a L, L′, L″, L¹, L², R¹, R^{1′}, R^{1″}, R², R^{2′}, and/or R^{2″} contain a substituent, selected from, for example, an alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, or thione group, or any combinations thereof.

In some embodiments, the bis(trialkoxysilyl) monomer is selected from, for example, 1,4-bis(trimethoxysilylethyl)benzene, 1,4-bis(trimethoxysilylmethyl)benzene, 1,4-bis(trimethoxysilyl)benzene, 1,4-bis(triethoxysilylethyl)benzene, 1,4-bis(triethoxysilyl)benzene, 1,2-bis(trimethoxysilylethyl)benzene, 1,2-bis(trimethoxysilylmethyl)benzene, 1,2-bis(trimethoxysilyl)benzene, 1,2-bis(triethoxysilylethyl)benzene, 1,2-bis(triethoxysilyl)benzene, 1,3-bis(trimethoxysilylethyl)benzene, 1,3-bis(trimethoxysilylmethyl)benzene, 1,3-bis(trimethoxysilyl)benzene, 1,3-bis(triethoxysilylethyl)benzene, 1,3-bis(triethoxysilyl)benzene, 4,4′-bis(triethoxysilyl)-1,1′-biphenyl, 4,4′-bis(trimethoxysilyl)-1,1′-biphenyl, 4,4′-bis(triethoxysilylmethyl)-1,1′-biphenyl, 4,4′-bis (trimethoxysilylmethyl)-1,1′-biphenyl, 4,4′-bis(triethoxysilylethyl)-1,1′-biphenyl, 4,4′-bis(trimethoxysilylethyl)-1,1′-biphenyl, 1,2-bis(triethoxysilyl)ethane, 1,2-bis(trimethoxysilyl)ethane, bis(triethoxysilyl)methane, bis(trimethoxysilyl)methane, bis[3-(trimethoxysilyl)propyl]amine, and bis[3-(triethoxy silyl)propyl]amine.

In some embodiments, a dialkyldialkoxysilane monomer of the polysiloxane matrix is of formula (II):

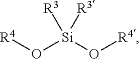

wherein R³, R^{3′}, R⁴, and R^{4′} are independently selected from CH₃, CH₂CH₃, and (CH₂)₂CH₃. In some embodiments, R³ and R^{3′} are identical. In some embodiments, R³ and R^{3′} are different. In some embodiments, R⁴ and R^{4″} are identical. In some embodiments, R⁴ and R^{4″} are different. In some embodiments, the dialkyldialkoxysilane monomer is selected from, for example, dimethyldimethoxysilane, diethyldimethoxysilane, and diethyldiethoxysilane.

In some embodiments, polysiloxane matrix comprises, for example, 1,4-bis(trimethoxysilylethyl)benzene and dimethyldimethoxysilane.

In some embodiments, the dialkyldialkoxysilane and bis(trialkoxysilyl) monomers are mixed in the presence of an acid, such as, sulfuric acid, nitric acid, hydrochloric acid, citric acid, and acetic acid.

In some embodiments, the polysiloxane matrix is formed from the dialkyldialkoxysilane and bis(trialkoxysilyl) monomers in the presence of one or more fluorophores (e.g., oxygen-sensitive fluorophore, oxygen-insensitive fluorophore, etc.), thereby embedding the fluorophores within the polysiloxane matrix.

In some embodiments, a polysiloxane matrix herein comprises an oxygen-sensitive fluorophore. In some embodiments, herein are oxygen-sensing materials (e.g., films) comprising an oxygen-sensitive fluorophore embedded within a polysiloxane matrix. In some embodiments, the oxygen-sensitive fluorophore is selected from one of the following (wherein M=Pt(II) or M=Pd(II)):

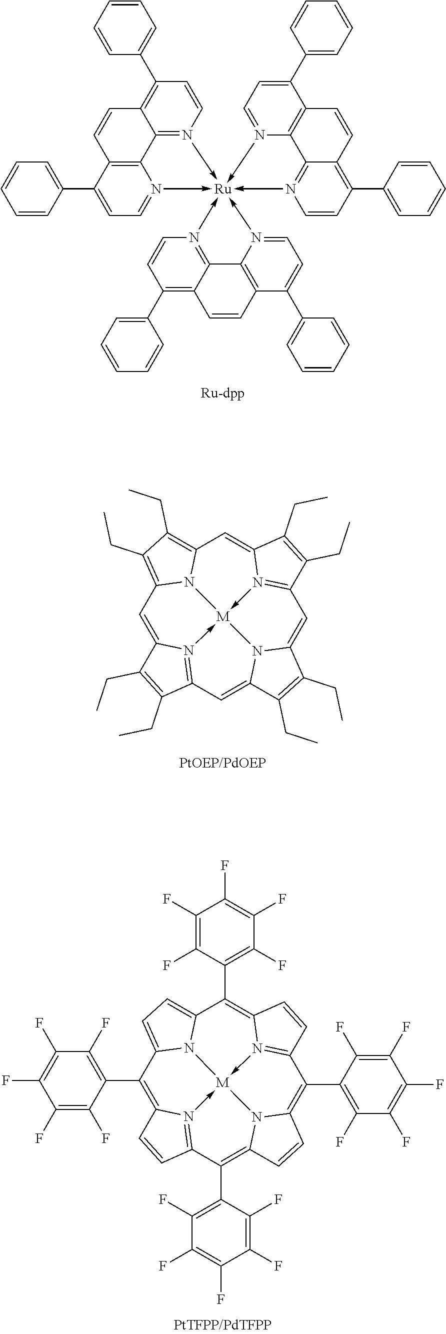

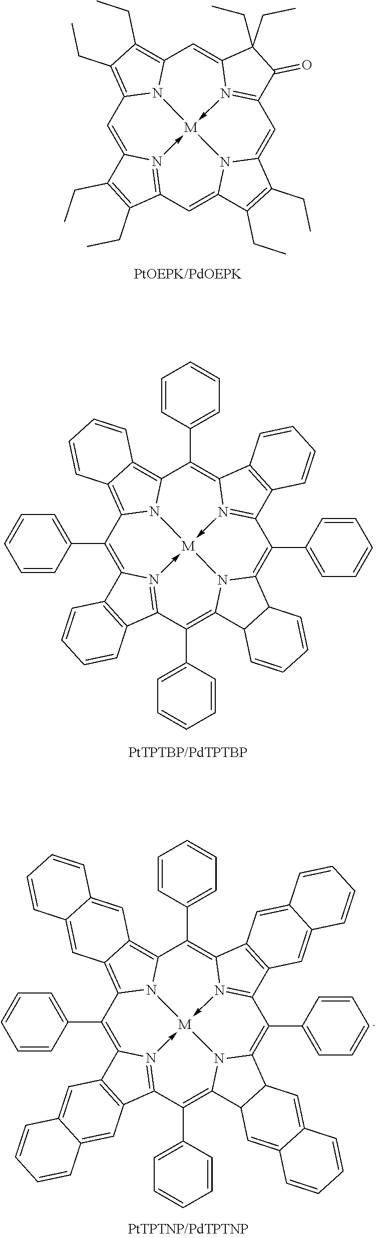

Other oxygen-sensitive fluorophores that are understood to those in the field are within the scope herein and may be included in the polysiloxane matrices described herein.

In some embodiments, a polysiloxane matrix herein comprises an oxygen-insensitive fluorophore. In some embodiments, the oxygen-insensitive fluorophore is embedded within the polysiloxane matrix (along with an oxygen-sensitive fluorophore). In some embodiments, herein are oxygen-sensing materials (e.g., films) comprising an oxygen-sensitive fluorophore and an oxygen-insensitive fluorophore embedded within a polysiloxane matrix, wherein the oxygen-sensitive fluorophore is used to monitor the oxygen concentration of a neighboring solution and the oxygen-insensitive fluorophore is used an oxygen-concentration-independent reference. Examples of oxygen-insensitive fluorophores that find use in embodiments herein include Coumarin-6, Nile blue chloride, tris(8-hydroxyquinolinato)aluminum (AlQ3), TAMRA, and fluorescein derivatives.

In some embodiments, a polysiloxane matrix herein comprises Coumarin-6 and PtTfPP.

In some embodiments, a polysiloxane matrix comprises a functionalized silane of formula (III):

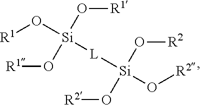

wherein R⁵ is a functional handle, such as a thiol, acrylate, methacrylate, maleimide, amine, COOH, azide, alkyne, or other click-chemistry groups, wherein L is a covalent bond, C₁-C₆ alkyl or heteroalkyl, C₂-C₆ alkenyl or heteroalkenyl, C₂-C₆ alkynyl or heteroalkynyl, and wherein R⁶, R^{6′}, and R^{6″} are independently selected from CH₃, CH₂CH₃, and (CH₂)₂CH₃. In some embodiments, R⁶, R^{6′}, and R^{6″} are identical. In some embodiments, R⁶, R^{6′}, and R^{6″} are different. An exemplary functionalized silane that may find use in embodiments herein is (3-Mercaptopropyl)methyldimethoxysilane:

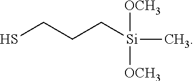

Other functionalized silanes with different L, R⁵, R⁶, R^{6′}, and R^{6″} are groups are within the scope herein. In some embodiments, the functionalized silane is included with the dialkyldialkoxysilane monomer and bis(trialkoxysilyl) monomer in the formation of the polysiloxane matrix. In some embodiments, a functionalized silane is added after initial formation of polysiloxane matrix to add chemical functionality, for example, on the surface of the polysiloxane matrix film. In some embodiments, the functional handle is used to attach proteins or small molecules covalently to the surface (e.g., environmentally sensitive fluorophores, biocompatible proteins, cells, etc.).

In some embodiments, a surface of a polysiloxane matrix is functionalized with a heterobifunctional compound that can be used for further functionalization of the surface. An exemplary heterobifunctional compound for use in surface functionalization of a polysiloxane matrix is Sulfo-SANPAH (sulfosuccinimidyl 6-(4′-azido-2′-nitrophenylamino)hexanoate):

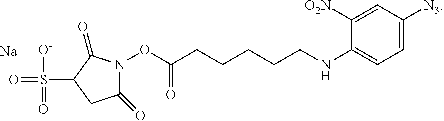 [SMILES: [Na+] ; [N-]=[N+]=Nc1ccc(NCCCCCC(=O)ON2C(=O)CC(S(=O)(=O)[O-])C2=O)c([N+](=O)[O-])c1]

In some embodiments, after formation of a polysiloxane matrix herein, the surface is functionalized with sulfo-SANPAH. The surface is first functionalized with reactive groups, followed by covalent conjugation of biomacromolecules to the surface. Specifically, the surface is exposed to the SANPAH and activated by UV light forming NHS moiety on the surface. Next, biomolecules and/or cells are introduced, and a covalent amide bond is formed between the surface and the biomolecules and/or cells.

In some embodiments, provided herein are films (e.g., thin layers) of the polysiloxane matrices (e.g., comprising oxygen-sensitive or oxygen-sensitive and oxygen-insensitive fluorophores) described herein. In some embodiments, a film herein is less than 5 mm in depth (e.g., 5 mm, 4 mm, 3 mm, 2 mm, 1 mm, 900 μm, 800 μm, 700 μm, 600 μm, 500 μm, or less or ranges therebetween). In some embodiments, a film is formed by depositing a layer of a desired thickness of a polysiloxane matrix herein (e.g., comprising an oxygen-sensitive fluorophore or oxygen-sensitive and oxygen-insensitive fluorophores) on a solid surface (e.g., a plate, bottom of a well, etc.).

In some embodiments, cell-interaction properties of a polysiloxane matrix film herein are enhanced by deposition of bioactive and/or biocompatible protein(s) and/or molecules atop the film. In some embodiments, the proteins/molecules are passively deposited onto the films. In other embodiments, the proteins/molecules are covalently or non-covalently conjugated to the polysiloxane matrix. In some embodiments, proteins/molecules are conjugated to the surface of a polysiloxane matrix film by a functionalized silane or heterobifunctional compound (e.g., SANPAH). In some embodiments, one or more bioactive extracellular matrix proteins are deposited/conjugated onto the film. Examples of suitable bioactive extracellular matrix proteins for use with the films herein include type I collagen, type IV collagen, fibronectin, laminin, vitronectin, and Matrigel. In some embodiments, one or more biocompatible molecules are deposited/conjugated onto the film. An example of a suitable biocompatible molecule for use with the films herein includes polydopamine.

In some embodiments, provided herein are methods of detecting the oxygen concentration within a solution. In some embodiments, methods are provided for determining an absolute oxygen concentration, while in other embodiments, methods are provided for determining the oxygen concentration relative to a reference or control. In certain embodiments, methods herein allow for the oxygen concentration to be monitored over time, thereby providing methods of monitoring the rate of change of the oxygen concentration in a sample.

In particular embodiments, methods are provided for monitoring the rate of oxygen consumption and/or production by a system. For example, cells consume oxygen as they respirate; therefore, monitoring the rate of oxygen depletion in a system comprising cells provides a method to monitor cellular metabolism.

In some embodiments, methods herein utilize the oxygen-sensitive fluorophores embedded within polysiloxane matrix, as described herein. When the oxygen-sensitive fluorophores are brought to an excited state by exposure to light at an appropriate excitation wavelength, they relax to a ground state by emitting light at an emission wavelength. The intensity of the emitted light is dependent upon the oxygen concentration. Therefore, the signal emitted from the fluorophores is a function of oxygen concentration. Monitoring the signal of the fluorophores allows one to monitor the oxygen concentration of a sample in contact with the polysiloxane matrix material.

In certain embodiments described herein, the polysiloxane matrix comprises both an oxygen-sensitive fluorophore and an oxygen-insensitive fluorophore. The oxygen-sensitive fluorophore functions as described in the preceding paragraph, changing emitted light intensity as a function of the adjacent oxygen concentration; however, the emitted light intensity of the oxygen-insensitive fluorophore remains constant, regardless of local oxygen concentration. The oxygen-insensitive fluorophore functions as a reference or control for accurately calculating the oxygen concentration.

As described below in the EXPERIMENTAL section, the oxygen concentration in a sample can be measured using the materials described herein, by application of the Stern-Volmer equation, which relates the relative fluorescence intensity of the oxygen-sensitive and an oxygen-insensitive fluorophores to the concentration of the oxygen.

$$[ {O}_{2} ] ( t ) = \frac{\frac{{I}_{0}}{I ( t )} - 1}{\frac{{I}_{0}}{{I}_{sat}} - 1} * {[ {O}_{2} ]}_{sat}$$

I₀ is a reference value equal to the ratiometric fluorescence intensity of the oxygen-sensitive fluorophore over the oxygen-insensitive fluorophore in the absence of oxygen. Iₛₐₜ is a reference value equal to the ratiometric fluorescence intensity of the oxygen-sensitive fluorophore over the oxygen-insensitive fluorophore at oxygen saturation. I(t) is a measured value equal to the ratiometric fluorescence intensity of the oxygen-sensitive fluorophore over the oxygen-insensitive fluorophore at time-point t. [O₂]ₛₐₜ is a reference value equal to the saturation concentration of oxygen. Using the Stern-Volmer equation, and having the necessary reference values in hand, allows a user to calculate the oxygen concentration at any time (t) in a sample or to monitor changes in oxygen concentration over a span of times.

In some embodiments, the polysiloxane-matrix-embedded fluorophore materials described herein are provided as films on solid surfaces in order to measure oxygen concentration of samples placed on those surfaces. In particular embodiments, methods and materials herein are used to measure oxygen consumption by cells in a sample. In such embodiments, it may be desirable for the cells to locate on are adjacent to the polysiloxane-matrix film. In such embodiments, bioactive/biocompatible protein/molecules are displayed on the sample-facing surface of the film. Such proteins/molecules promote the congregation of cells along the film surface. Monitoring oxygen concentration over time in such a system allows for monitoring the rate of oxygen consumption of the cells.

In some embodiments, the films herein are placed on the bottom of the wells of a 96-well or 384-well plate. In such a system, the oxygen concentration can be determined for multiple samples (e.g., containing cells) of various conditions using a standard laboratory plate reader.

### EXPERIMENTAL

Experiments were conducted during development of embodiments herein to fabricate and test an exemplary device composed of a siloxane matrix containing two dyes, platinum(II) meso-(2,3,4,5,6-penta- fluoro)phenyl porphyrin (PtTFPP) and 3-(2-Benzothiazolyl)-N,N-diethylumbelliferylamine (Coumarin-6). The cellular interface of the siloxane matrix was functionalized with a biocompatible layer comprising Collagen I. The dye-impregnated siloxane matrix was deposited onto a glass substrate.

To fabricate the oxygen sensitive matrix, bis(trimethoxysilylethyl)benzene (18.8% v/v) was mixed with dimethyldiethoxysilane (37.6% v/v), 0.1N hydrochloric acid (5.6% v/v), PtTFPP (1.5 mg mL-1 in ethanol, 37.6% v/v), and Coumarin-6 (10 mg mL-1 in THF, 0.2% v/v). The mixture was stirred for 3 hours, then deposited onto a substrate, air-dried 24 hours, and oven cured at 60° C. overnight. The substrate was not washed.

After silane film fabrication, the film surface was further modified with a biomacromolecule coating to enhance cell attachment. Biomacromolecules assessed for cell attachment include collagen type I (rat tail, acid extracted) and laminin. Passive coating of the collagen type I was determined to be sufficient for cell attachment. Passive coating of the substrate is achieved by incubating a solution of the biomacromolecule (e.g., 300 ug mL-1 collagen type I in 17 mM acetic acid) on the film for either 1 hour at room temperature or overnight at 4° C. Excess biomacromolecule was removed by washing the substrate with phosphate buffered saline (PBS).

The oxygen sensitive component of the invention is the PtTFPP compound. Molecular oxygen quenches the phosphorescence of PtTFPP, such that phosphorescence is low in high oxygen conditions, and vice versa. The oxygen concentration in the film is calculated from the Stern-Volmer equation, which relates the relative phosphorescence intensity of PtTFPP to the concentration of the oxygen. Here, I₀ is the ratiometric fluorescence intensity (650 nm/490 nm) of the film in the absence of oxygen. Iₛₐₜ is the ratiometric fluorescence intensity of the film at oxygen saturation.

$$[ {O}_{2} ] ( t ) = \frac{\frac{{I}_{0}}{I ( t )} - 1}{\frac{{I}_{0}}{{I}_{sat}} - 1} * {[ {O}_{2} ]}_{sat}$$

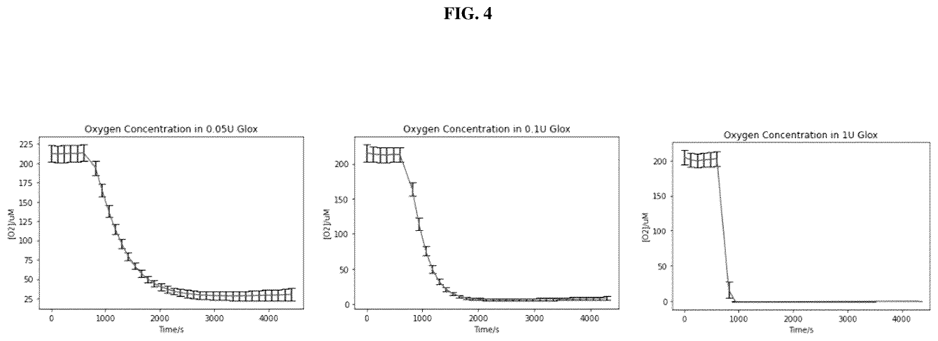

The chemistry was evaluated first using a glucose oxidase/glucose biochemical assay. Glucose oxidase can consume glucose and O₂ to create different dissolved O₂ concentrations in the solution. Different concentrations of glucose oxidase (0.05-1 U/well) were added to the wells of a plate with the oxygen sensing film immobilized at the bottom of the well. Glucose was then added, and the fluorescence intensity of the oxygen sensing film was measured for 1-2 hr. The dissolved oxygen concentration in the solution was calculated based on the Stern-Volmer equation. The different oxygen consumption rates were observed and were found to be consistent with the concentration of glucose oxidase added. In addition, the data was reproducible with small error bars in FIG. 4.

The surface was coated with Collagen I and Laminin, either covalently or passively. Covalent conjugation is achieved with sulfo-SANPAH conjugation method, and passive coating is achieved by incubating the siloxane surface in the presence of the protein for 1 hour at room temperature. The protein was titrated from 100 ug mL-1-3,000 ug mL-1. C2C12 cells were seeded on the coated surface at 60,000 cells/cm², and cellular coverage on the substrate was assessed the following day. It was found that coating the substrate with Collagen I and Laminin enhanced cellular coverage on the substrate, but there was no difference between the covalent and passive coatings. Results also showed that 300 ug mL-1 collagen I and laminin were sufficient to enhance the adhesion of cells to the surface.

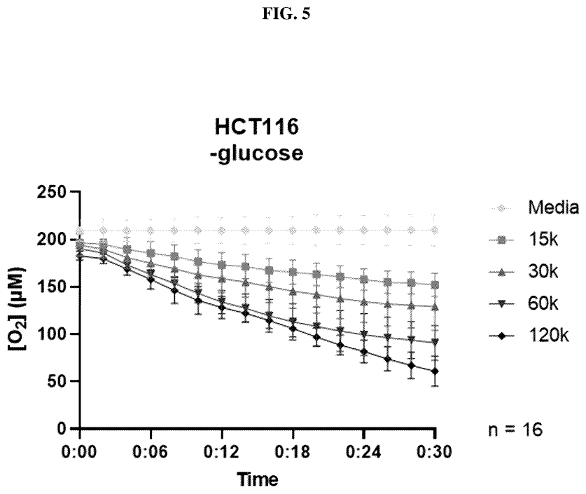

Successful oxygen consumption experiments were conducted with HCT-116 cell line. To determine the minimum number of cells necessary to observe a signal change, HCT-116 cells were titrated from 15k-120k cells/well (95k-750k cells/cm²). The following day, the culture medium of the cells was replaced with fresh DMEM (high glucose, without phenol red), and the ratiometric fluorescence of 650/490 nm was recorded over time in a plate reader with temperature control at 37° C. Results showed that as cell density increased, the oxygen concentration decreased more rapidly (oxygen consumption rate, OCR) (FIG. 5). Experiments determined that at 40,000 cells/well (250k/cm²), the ratiometric signal increased sufficiently over two hours for oxygen consumption measurements. Further experiments using drug treatments were carried out at 250k cells/cm².

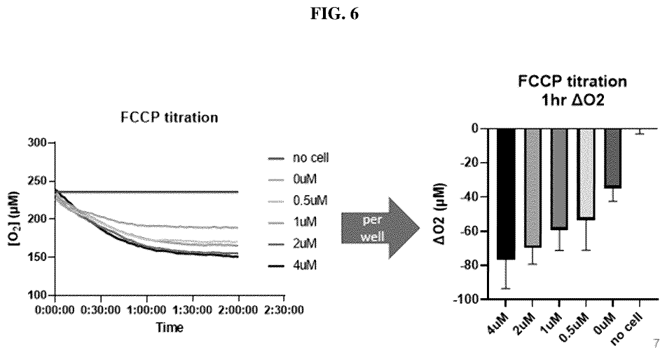

HCT-116 cells were seeded onto the oxygen sensing surface functionalized with 300 ug mL-1 Collagen I at 40,000 cells/well of a half-area 96-well plate (250,000 cells/cm²). The following day, the culture medium was replaced with medium containing 2-[[4-(trifluoromethoxy)phenyl]hydrazinylidene]propanedinitrile (FCCP), an uncoupler of oxidative phosphorylation in mitochondria that increases oxygen consumption. The results showed that oxygen consumption increased with increasing FCCP concentration up to 4 uM (FIG. 6, top panels). HCT-116 cells were additionally treated with rotenone at 2 uM, which is an inhibitor of oxidative phosphorylation. Results showed that rotenone treatment inhibited oxygen consumption in these cells (FIG. 6, bottom panel).

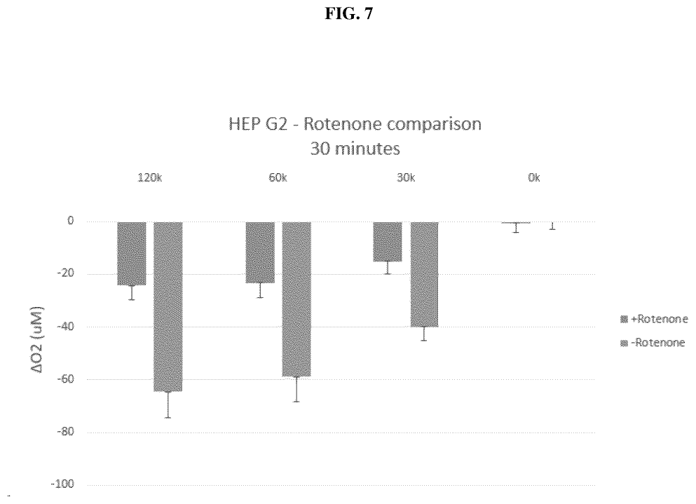

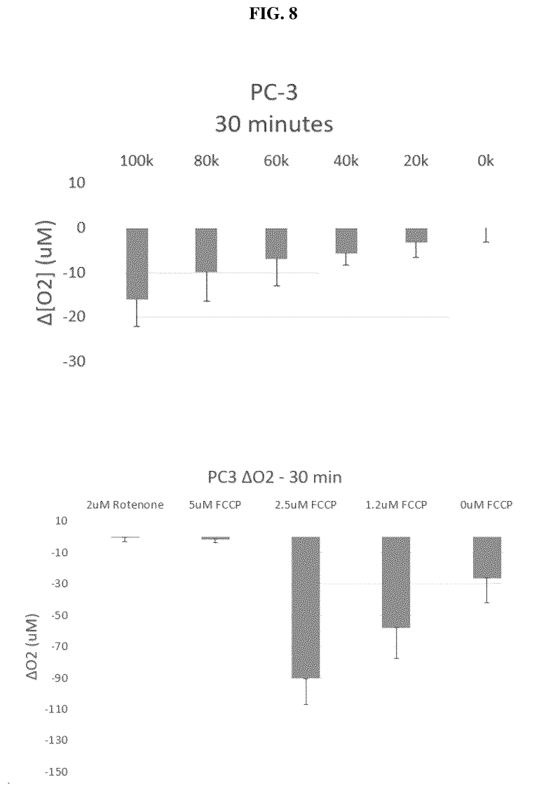

Similar tests were carried out with additional cell lines HEP-G2 (FIG. 7) and PC-3 (FIG. 8). Results obtained with these cells also showed increased oxygen consumption with increasing cell density. Similarly, FCCP treatment increased oxygen consumption and rotenone treatment decreased oxygen consumption for HEP-G2 & PC-3.

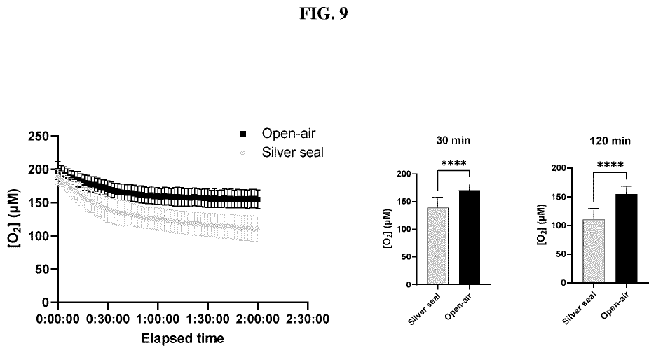

Experiments conducted during development of embodiments herein demonstrated that the use of an oxygen barrier, in the form of aluminum foil over the well containing the assay reagents, further enhance the rate change of oxygen observed with this invention (FIG. 9).

1. A polysiloxane matrix comprising:
(a) a bis(trialkoxysilyl) monomer of formula (I): 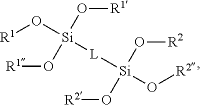
wherein R¹, R^{1′}, R^{1″}, R², R^{2′}, and R^{2″} are independently selected from CH₃, CH₂CH₃, (CH₂)₂CH₃, wherein L is selected from C₁-C₆ alkyl, C₁-C₆ heteroalkyl, C₂-C₆ alkenyl, C₂-C₆ heteroalkenyl, C₂-C₆ alkynyl, C₁-C₆ heteroalkynyl, 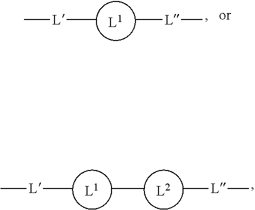
wherein L′ and L″ are independently selected from, a covalent bond, C₁-C₆ alkyl or heteroalkyl, C₂-C₆ alkenyl or heteroalkenyl, C₂-C₆ alkynyl or heteroalkynyl, wherein L¹ and L² are independently selected aryl or heteroaryl rings, wherein L¹ and L² are linked by a covalent bond, and wherein L, L′, L″, and L¹ and L² are optionally branched or substituted;
(b) a dialkyldialkoxysilane monomer of formula (II): 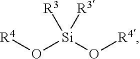
wherein R³, R^{3′}, R⁴, and R^{4′} are independently selected from CH₃, CH₂CH₃, and (CH₂)₂CH₃.

2. The polysiloxane matrix of claim 1, wherein:
(i) each of R¹, R^{1′}, R^{1″}, R², R^{2′}, and R^{2″} are the same;
(ii) L′ and L″, when present, are the same;
(iii) L¹ and L², when present, are the same;
(iv) R³ and R^{3′} are the same; and/or
(v) R⁴ and R^{4″} are the same.

3. The polysiloxane matrix of claim 1, wherein R¹, R^{′}, R^{1″}, R², R^{2′}, and R^{2″} are CH₃ or CH₂CH₃.

4. The polysiloxane matrix of claim 1, wherein L is selected from (CH₂)₁₋₆, (CH₂)₁₋₆—NH—(CH₂)₁₋₆, (CH₂)₁₋₆—O—(CH₂)₁₋₆, (CH₂)₀₋₆-aryl-(CH₂)₁₋₆, and (CH₂)₁₋-aryl-aryl-(CH₂)₁₋₆.

5. The polysiloxane matrix of claim 1, wherein the bis(trialkoxysilyl) monomer is selected from 1,4-bis(trimethoxysilylethyl)benzene, 1,4-bis(trimethoxysilylmethyl)benzene, 1,4-bis(trimethoxysilyl)benzene, 4,4′-bis(triethoxysilyl)-1,1′-biphenyl, 1,2-bis(triethoxysilyl)ethane, and bis[3-(trimethoxysilyl)propyl]amine.

6. The polysiloxane matrix of claim 1, wherein R³, R^{3′}, R⁴, R^{4′} are CH₃ or CH₂CH₃.

7. The polysiloxane matrix of claim 1, wherein the bis(trialkoxysilyl) monomer is 1,4-bis(trimethoxysilylethyl)benzene and the dialkyldialkoxysilane monomer is dimethyldimethoxysilane.

8. A composition comprising the polysiloxane matrix of claim 1 and an oxygen-sensitive fluorophore.

9. The composition of claim 8, wherein the oxygen-sensitive fluorophore is selected from tris(4,7-diphenyl-1,10-phenanthroline) ruthenium(II) chloride (Ru-dpp), Platinum octaethylporphyrin; platinum(II) 2,3,7,8,12,13,17,18-octaethyl-21H,23H-porphyrin (PtOEP), Palladium(II) octaethylporphine (PdOEP), Platinum(II)-5,10,15,20-tetrakis-(2,3,4,5,6-pentafluorphenyl)-porphyrin (PtTfPP), palladium(II)-5,10,15,20-tetrakis-(2,3,4,5,6-pentafluorphenyl)-porphyrin (PdTFPP), platinum(II) octaethylporphyrinketone (PtOEPK), palladium(II) octaethylporphyrinketone (PdOEPK), platinum(II) tetraphenyltetrabenzoporphyrin (PtTPTBP), meso-Tetraphenyl-tetrabenzoporphine Palladium Complex (PdTPTBP), platinum(II) tetraphenyltetranaphthoporphyrin (PtTPTNP), and palladium(II) tetraphenyltetranaphthoporphyrin (PdTPTNP).

10. The composition of claim 8, further comprising an oxygen-insensitive fluorophore.

11. The composition of claim 10, wherein the oxygen-insensitive fluorophore is selected from Nile blue chloride, tris(8-hydroxyquinolinato)aluminum (AlQ3), TAMRA, and Coumarin-6.

12. The composition of claim 10, wherein the oxygen-insensitive fluorophore is Coumarin-6 and the oxygen-sensitive fluorophore is PtTfPP.

13. A thin film comprising the composition of claim 8.

14. A device comprising a solid surface with a thin film of claim 13 deposited thereupon.

15. The device of claim 14, wherein the solid surface is a glass or polystyrene surface.

16. (canceled)

17. The device of claim 14, further comprising a layer of bioactive extracellular matrix protein or biocompatible molecules on top of the thin film.

18. The device of claim 17, wherein the layer of bioactive extracellular matrix protein comprises one or more proteins selected from type I collagen, type IV collagen, fibronectin, and laminin.

19. The device of claim 17, wherein the layer of biocompatible molecules comprises polydopamine.

20. (canceled)

21. The device of claim 17, wherein the bioactive extracellular matrix protein is covalently or non-covalently attached to the thin film.

22. A method comprising:
(a) contacting (i) an oxygen-sensitive fluorophore embedded within a polysiloxane-matrix with (ii) a test solution;
(b) exposing the oxygen-sensitive fluorophore to light within the excitation spectrum of the oxygen-sensitive fluorophore;
(c) detecting light within the emission spectrum of the oxygen-sensitive fluorophore;
(d) determining the concentration of oxygen in the test solution.

23-47. (canceled)
